(19) United States
(12) Patent Application Publication
(10) Pub. No.: US 2014/0150795 A1
(43) Pub. Date: Jun. 5, 2014

(54) SYSTEM AND METHOD FOR DETECTING DOUBLE TRIGGERING WITH REMOTE MONITORING

(71) Applicant: COVIDIEN LP, Mansfield, MA (US)

(72) Inventor: Gary Milne, Louisville, CO (US)

(73) Assignee: COVIDIEN LP, Mansfield, MA (US)

(21) Appl. No.: 13/690,618

(22) Filed: Nov. 30, 2012

(51) Int. Cl. A61M 16/00

(52) U.S. Cl. CPC A61M 16/0057
USPC 128/205.23

(57) ABSTRACT

Methods and systems are provided that determine whether a patient is asynchronous with a ventilator. In certain embodiments, a ventilation system may determine whether a double-triggering event has occurred between the patient and the ventilator. The ventilation system may compare a value of the patient's exhaled tidal volume to a threshold and may determine that a double-triggering event occurred if the patient's exhaled tidal volume is less than or equal to the threshold. The ventilation system may also determine a frequency of the double-triggering events. Further, the ventilation system may provide an indication of a detected double-triggering event and an indication of the frequency of double-triggering events.

### BACKGROUND

The present disclosure relates generally to medical devices, and more particularly, to medical devices that provide respiratory support to a patient, such as ventilators.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a medical device may be used to control the flow of air, foods, fluids, or other substances to the patient. For example, ventilators may be used to provide supplemental oxygen support to the patient. Such ventilators typically include a source of pressurized oxygen, which may be delivered to the patient through a conduit. The ventilators may also monitor and display one or more breathing characteristics of the patient during ventilation. The ventilators may use the monitored one or more breathing characteristics to determine appropriate ventilation parameters for the patient. Additionally, a caregiver may evaluate the one or more breathing characteristics to adjust the ventilation parameters set by the ventilator.

In particular, the caregiver may evaluate the one or more breathing characteristics to increase the patient's comfort and/or to decrease the patient's work of breathing. For example, the caregiver may wish to monitor the degree of asynchrony between the patient and the ventilator. Asynchrony may occur when the patient's neural inspiratory time (i.e., the patient's desired inspiratory time) differs from the mechanical inspiratory time set by the ventilator. Double-triggering, which is a type of asynchrony, may occur when the patient's neural inspiratory time is greater than the preset mechanical inspiratory time and/or when the patient's desired flow is greater than the preset flow. Specifically, the patient may continue to inhale beyond the preset mechanical inspiratory time and may trigger a second breath (e.g., a stacked breath) without exhaling. Essentially, the ventilator delivers two breaths to the patient in response to a single patient effort. Accordingly, it may be desirable to monitor and decrease the occurrence of double-triggering between the patient and the ventilator to increase the patient's comfort and/or to decrease the patient's work of breathing. Unfortunately, double-triggering events may be difficult for the caregiver to recognize and/or the frequency of double-triggering over a period of time may be difficult for the caregiver to assess.

### BRIEF DESCRIPTION OF THE DRAWINGS

Advantages of the disclosed techniques may become apparent upon reading the following detailed description and upon reference to the drawings in which:

FIG. 1 is a block diagram of ventilation system including a ventilator in accordance with an embodiment;

FIG. 2 is a flow diagram of a method for providing an indication of double-triggering event detected using the ventilation system of FIG. 1 in accordance with an embodiment;

FIG. 3 is an illustration of a display of the ventilator of FIG. 1 including an indication of a double triggering event detected in accordance with an embodiment;

FIG. 4 is an illustration of a display of the ventilator of FIG. 1 including a graphical representation of a patient's inspiration in accordance with an embodiment;

FIG. 5 is an illustration of a display of the ventilator of FIG. 1 including a graphical representation of a patient's inspiration and an indication of a double-triggering event in accordance with an embodiment;

FIG. 6 is an illustration of a display of the ventilator of FIG. 1 including a graphical representation of a patient's inspiration and an indication of a double-triggering event in accordance with an embodiment;

FIG. 7 is an illustration of a display of the ventilator of FIG. 1 including a graphical representation of a frequency of double-triggering in accordance with an embodiment;

FIG. 8 is an illustration of a display of the ventilator of FIG. 1 including a graphical representation of a frequency of double-triggering and an indication of double-triggering detected in accordance with an embodiment;

FIG. 9 is an illustration of a display of the ventilator of FIG. 1 including an indication of double-triggering detected in accordance with an embodiment; and

FIG. 10 is an illustration of a display of the ventilator of FIG. 1 including an indication of an indication of historical data relating to double-triggering events in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, a ventilator may monitor one or more breathing characteristics of a patient and use the breathing characteristics to set and adjust one or more ventilation parameters. In particular, certain ventilators may use the monitored one or more breathing characteristics to identify when the patient is triggering a breath. Specifically, for a ventilator operating under a triggered mode (i.e., spontaneous breaths only) or a hybrid mode (i.e., mandatory and spontaneous breaths), the ventilator may be configured to monitor the pressure and/or flow in the patient's airway and/or the respiratory circuit to determine whether the patient is triggering a breath. Accordingly, the ventilator may deliver a breath to the patient in response to determining that the patient is triggering a breath. However, in some instances, the patient may inadvertently trigger a second breath from the ventilator. For example, if the patient desires a breath with a greater volume or a longer inspiration time than is set by the ventilator and the patient continues to exert effort to breathe, the ventilator may deliver a second breath to the patient without allowing the patient to sufficiently expire. This occurrence is generally referred to as a double-trigger or a stacked breath. In some instances, the patient may briefly expire before the second breath, but the duration and/or flow of the expiration may not be sufficient to achieve a desired emptying of the lungs. Alternatively, the patient may not expire at all in between the first and second breaths. As noted above, it may be desirable to reduce the occurrence of double-triggering to increase the patient's comfort and/or to decrease the patient's work of breathing. Unfortunately, double-triggering events may be difficult for the caregiver to recognize and/or the frequency of double-triggering over a period of time may be difficult for the caregiver to assess.

Accordingly, the disclosed embodiments provide a system and method for identifying double-triggering events and providing indications to a caregiver regarding the occurrence of a double-trigger event and/or the frequency of double-triggering over a period of time. For example, a ventilator may be configured to monitor the volume of exhaled breaths (e.g., the exhaled tidal volume) of a patient. The ventilator may compare the exhaled tidal volume to a threshold to determine whether a double-triggering event occurred. In particular, ventilator may determine that a double-triggering event occurred if the exhaled tidal volume is approximately equal to zero milliliters. Additionally, the ventilator may be configured to provide an indication of the detected double-triggering event to alert a caregiver to the occurrence of each double-triggering event. As will be described in more detail below, the indication of a double-triggering event detected may include an audible indication, a graphical indication, and/or a textual indication.

In certain embodiments, the ventilator may additionally determine whether a frequency of double-triggering is above a frequency threshold. For example, the ventilator may determine the number of double-triggering events within a predetermined period of time, the percentage of double-triggering events out of the total number of breaths within a predetermined period of time, the total number of double-triggering events, and/or the total percentage of double-triggering events out of the total number of breaths. Accordingly, each value of the frequency may be compared to a respective frequency threshold. Further, the ventilator may provide an indication of the frequency of double-triggering when the frequency of double-triggering is above a respective frequency threshold. Comparing the frequency of double-triggering to a frequency threshold may be desirable for various circumstances. In particular, the frequency of double-triggering may be representative of the severity of double-triggering. Thus, alerting the caregiver when the frequency exceeds a respective frequency threshold may provide an indication to the caregiver to take steps to reduce the frequency of double-triggering. As will be described in more detail below, the indication of the frequency of double-triggering may include an audible indication, a graphical indication, and/or a textual indication.

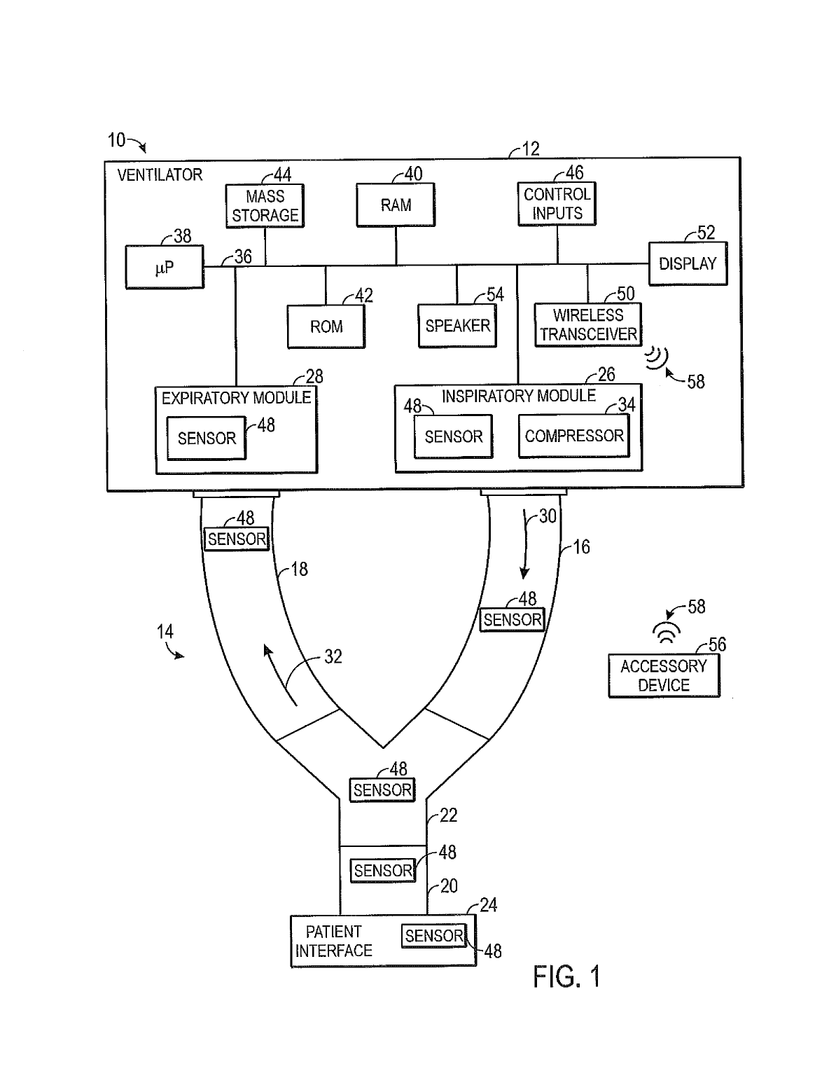

With the foregoing in mind, FIG. 1 illustrates a ventilation system 10 for providing respiratory support to a patient. The ventilation system 10 may include a ventilator 12 connected to a respiratory circuit 14. The respiratory circuit 14 may be in fluid communication with a source of respiratory gas and may enable one-way flow of inspired gases towards the patient and one-way flow of expired gases away from the patient. In particular, the respiratory circuit 14 may include an inspiratory conduit 16, an expiratory conduit 18, and a patient conduit 20. The inspiratory, expiratory, and patient conduits 16, 18, and 20, may be connected to one another by a Y-connector (i.e., a “wye” connector) 22, which may be connected to a patient interface 24. The patient interface 24 may be any suitable patient interface, such as an endotracheal tube, a tracheostomy tube, or a breathing mask placed over the nose and/or mouth of the patient. Furthermore, the system 10 may include any number of connectors or medical tubing to provide the respiratory gas from the source to the lungs.

The ventilator 12 may include an inspiratory module 26 and an expiratory module 28 for circulating respiratory gases to and from the patient via the respiratory circuit 14 and the patient interface 24. Accordingly, the inspiratory module 26 may be coupled to the inspiratory conduit 16 for providing respiratory gases, represented by arrow 30, and the expiratory module 28 may be coupled to the expiratory conduit 18 for receiving respiratory gases, represented by arrow 32. As used herein, the respiratory gas may be air, oxygen, nitrogen, carbon dioxide, vaporized water, vaporized medicines, or any combination thereof. The inspiratory module 26 may be configured to receive a source of respiratory gas and to pressurize the respiratory gas via a compressor 34. Additionally or alternatively, the inspiratory module 26 may receive a source of pressurized respiratory gas, such as a compressed air wall outlet or a tank of pressurized respiratory gas. Furthermore, the inspiratory and expiratory modules 26 and 28 may include various suitable components, such as circuitry, valves, filters, tubing, and/or sensors. In one embodiment, the inspiratory and expiratory modules 26 and 28 may be coupled to an internal bus 36 and controlled by a processor 38 to regulate the pressure and/or flow of the respiratory gas delivered and removed. The processor 38 may be configured to control the operation of the inspiratory and expiratory modules 26 and 28 based at least in part upon a ventilator operating mode, such as a triggered mode or a hybrid mode. In a triggered mode, the inspiratory module 26 may be configured to deliver spontaneous breaths to a patient. As defined herein, a spontaneous breath is ventilation support that is delivered in response to a patient trigger. In a hybrid mode, the inspiratory module 26 may be configured to deliver both mandatory and spontaneous breaths. As defined herein, a mandatory breath is ventilation support that is delivered in response to a mandatory pattern (i.e., time-triggered).

The processor 38 may access and execute coded instructions, such as for implementing the algorithms discussed herein, from one or more storage components of the ventilator 12, such as a RAM 40, ROM 42, and/or a mass storage device 44. For example, code encoding executable algorithms may be stored in the RAM 40, the ROM 42, and/or the mass storage device 44 (such as a magnetic or solid state hard drive or memory or an optical disk or memory) and accessed and operated according to processor 38 instructions using stored data. In certain embodiments, the RAM 40, the ROM 42, and/or the mass storage device 44 may store information related to one or more settings of the ventilator 12, one or more coefficients or equations for calculating patient physiological parameters, and patient data. For example, patient data such as normal values or ranges of respiratory resistance and compliance for various patient populations may be stored. The processor 38 may also receive information related to ventilation settings and/or patient data from a caregiver via one or more control inputs 46. For example, a caregiver may input a patient's gender, age, weight, ideal body weight, and/or condition (e.g., asthma, emphysema, chronic obstructive pulmonary disease, acute respiratory distress syndrome, etc.) which may be used in the selection of the normal values of respiratory resistance and compliance, as well as alarm conditions. Additionally, the processor 38 may receive information from one or more sensors 48 of the ventilation system 10, as will be described in more detail below. In certain embodiments, the processor 38 may also receive information related to patient physiological parameters from other medical devices (e.g., a pulse oximeter, an electrocardiography device, an end-tidal carbon dioxide (EtCo₂) monitor, and/or an electroencephalogy (EEG) device) via a wireless transceiver 50. The received information may be stored in the RAM 40, the ROM 42, and/or the mass storage device 44 and may be used in calculations for determining one or more physiological parameters of a patient relating to respiratory function. The ventilator 12 may also include a display 52 and/or a speaker 54, which may be used to convey information about the calculated physiological parameters and/or ventilation parameters or settings to the caregiver. Furthermore, the wireless transceiver 50 may be configured to transmit information to one or more accessory devices 56 via wireless communication 58 to enable the caregiver to remotely monitor the calculated physiological parameters and/or the ventilation parameters. For example, the one or more accessory devices 56 may include a remote computer (e.g., located at a nurse's station), a pager, a smart phone, a smart watch, a laptop computer, a handheld computing device, or a cloud computing device.

As noted above, the processor 38 may calculate the one or more physiological parameters based in part upon signals received from the one or more sensors 48 in the ventilation system 10. The sensors 48 may obtain signals related to the flow and/or pressure of the supplied and returned respiratory gases, which may be indicative of the patient's respiratory function and in particular, of the patient's effort (i.e., a patient trigger). Accordingly, any suitable sensor 48 for determining flow, pressure, nerve impulses, concentrations of components in the patient's respiratory gas, or any other desired parameter may be used. For example, the sensors 48 may be pressure sensors, flow sensors, electroencephalogy (EEG) sensors, neural sensors, and/or optical sensors. Additionally, the sensors 48 may generate signals related to certain physiological parameters, such as pressure and flow, which may be used by the processor 38 to derive other physiological parameters. For example, the processor 38 may be configured to derive exhaled tidal volume, inhaled tidal volume, inspiratory time, expiratory time, a ratio of inspiratory time to expiratory time (I:E), respiratory rate, peak inspiratory pressure, positive end-expiratory pressure, plateau pressure, alveolar pressure, inspiratory reserve volume, expiratory reserve volume, vital capacity, functional residual capacity, respiratory resistance, respiratory compliance, and/or any other physiological parameter. In particular, the processor 38 may be configured to integrate the determined inspiratory flow and expiratory flow to derive the inhaled tidal volume and exhaled tidal volume, respectively. Additionally, the processor 38 may be configured to graphically represent one or more physiological parameters on the display 52 and to provide visual or audible indications related to one or more physiological parameters via the display 52 or the speaker 54, which will be described in more detail below.

The sensors 48 may be placed at any suitable location for measuring physiological parameters of the patient. For example, the ventilation system 10 may include sensors 48 located within the ventilator 12, such as within the inspiratory and the expiratory modules 26 and 28. Additionally, sensors 48 may be disposed about the respiratory circuit 14. In certain embodiments, inspiratory, expiratory, and patient conduits 16, 18, and 20 and the Y-connector 22 may each include one or more sensors 48. Additionally, it may be desirable to obtain measurements near the lungs and/or near the diaphragm of the patient. As such, one or more sensors 48 may be located within or disposed about the patient interface 24. In certain embodiments, in an effort to reduce the dead space volume within the respiratory circuit 14 and/or the patient interface 24, the sensors 48 may be embedded in the walls of the inspiratory, expiratory, and patient conduits 16, 18, and 20, the Y-connector 22, and/or the walls of the patient interface 24 (e.g., the walls of an endotracheal or tracheostomy tube). Additionally, it should be noted that one or more lead wires (not shown) may couple the sensors 48 to the ventilator 12 to power the sensors 48 and transmit the signals.

As noted above, the processor 38 may be configured to cause the inspiratory and expiratory modules 26 and 28 to operate under a triggered mode or a hybrid mode. As noted above, in a triggered mode, the inspiratory module 26 is triggered to deliver spontaneous breaths to the patient by the patient's inspiratory muscles (i.e., a patient trigger). In particular, the processor 38 may be configured to detect a decrease in pressure and/or flow in the patient's airway, which may be indicative of a patient trigger, based at least in part upon received signals from the sensors 48. Additionally, the inspiratory and expiratory modules 26 and 28 may be configured to terminate the delivered breath and transition to expiration based at least in part upon a preset inspiratory time, inhaled tidal volume, and/or respiratory rate, and may be at least partially dependent upon the mode of ventilation. The triggered mode of ventilation may be a volume support mode, a pressure support mode, a proportional pressure support mode, a continuous positive airway pressure mode, an assisted spontaneous breathing mode, or a spontaneous breathing mode. In certain embodiments, the caregiver may select or input the triggered mode of ventilation and/or one or more ventilation parameters (e.g., inspiratory time, inhaled tidal volume, respiratory rate, etc.).

In a hybrid mode, the inspiratory module 26 may be configured to deliver both mandatory and spontaneous breaths. As described above, the processor 38 may detect a decrease in pressure and/or flow in the patient's airway to detect the occurrence of a patient trigger and may cause the inspiratory module 26 to deliver a spontaneous breath in response to the patient trigger. Additionally, the inspiratory module 26 may deliver mandatory breaths, which may be based at least in part upon a preset respiratory rate. Similar to a triggered mode of ventilation, the inspiratory and expiratory modules 26 and 28 may be configured to terminate the delivered breath and transition to expiration based at least in part upon a preset inspiratory time, inhaled tidal volume, and/or respiratory rate, and may be at least partially dependent upon the mode of ventilation. The hybrid mode of ventilation may be an intermittent positive-pressure ventilation mode, a synchronized controlled mandatory ventilation mode, a volume control mode, a volume assist-control mode, a volume control plus mode, a biphasic positive airway pressure mode, a pressure controlled mandatory ventilation mode, a pressure assist-control mode, a pressure control mode, an adaptive pressure ventilation mode, a pressure-regulated volume control mode, or a pressure-regulated volume control assist mode. In certain embodiments, the caregiver may select or input the hybrid mode of ventilation and/or one or more ventilation parameters (e.g., inspiratory time, inhaled tidal volume, respiratory rate, etc.).

Unfortunately, in some situations, the preset inspiratory time, inhaled tidal volume, and/or respiratory rate may differ from the patient's desired inspiratory time, inhaled tidal volume, and/or respiratory rate. For example, the inspiratory and expiratory modules 26 and 28 may transition to expiration earlier than the patient desires. In response, the patient may continue trying to breathe after the termination of the inspiratory period. Thus, the processor 38 may detect a decrease in pressure and/or flow in the patient's airway and may determine that the patient is triggering a breath. In this manner, the inspiratory module 26 may deliver a second breath (e.g., a double-trigger or a stacked breath) to the patient. As noted above, it may be desirable to reduce the occurrence of double-triggering to increase the patient's comfort and/or to decrease the patient's work of breathing. Unfortunately, double-triggering events may be difficult for the caregiver to recognize and/or the frequency of double-triggering over a period of time may be difficult for the caregiver to assess. Accordingly, it may be desirable to detect double-triggering events and to provide an indication when a frequency of double-triggering exceeds a frequency threshold.

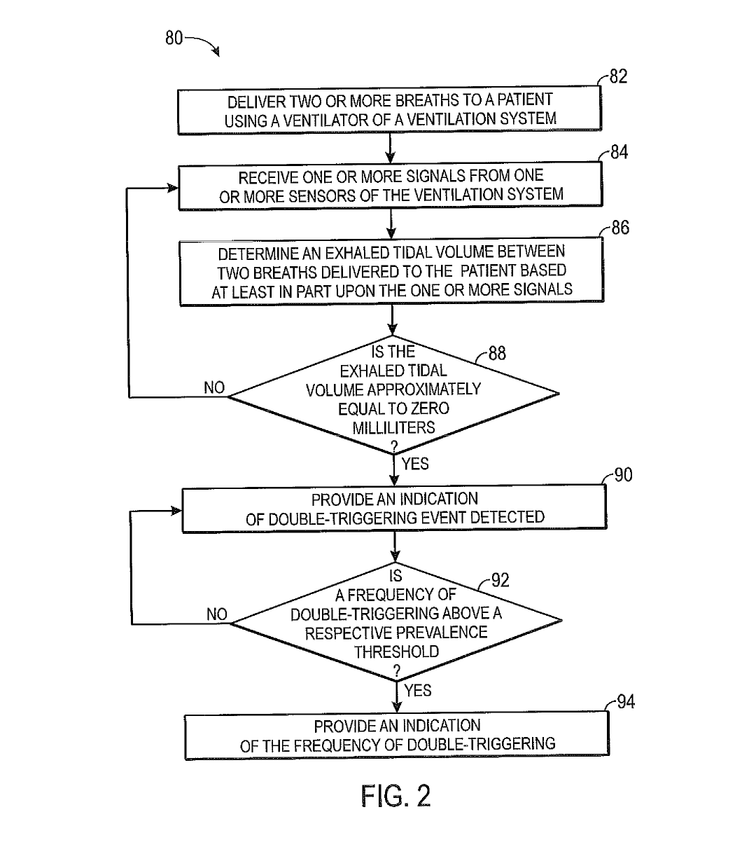

With the foregoing in mind, FIG. 2 illustrates a method 80 for detecting double-triggering events in accordance with some embodiments. The method 80 may be performed as an automated procedure by a system, such as the ventilation system 10. In addition, certain steps of the method 80 may be performed by a processor, or a processor-based device such as the ventilator 12 that includes instructions for implementing certain steps of the method 80.

The method 80 may include delivering two or more breaths to a patient using a ventilator of a ventilation system (e.g., the ventilator 12 of the ventilation system 10) (block 82). As described above, the ventilator 12 may be operating in a triggered mode or a hybrid mode. Thus, the two or more breaths may be spontaneous breaths, mandatory breaths, or a combination thereof. The method 80 may also include receiving one or more signals from one or more sensors, such as the sensors 48, of the ventilation system 10 (block 84). For example, the processor 38 may receive the signals from the sensors 48 via one or more leads. The processor 38 may process and/or analyze the signals and may utilize information from the signals to detect patient triggers and/or to determine one or more physiological parameters of the patient. In particular, the processor 38 may determine an exhaled tidal volume between two breaths delivered to the patient based at least in part upon the one or more signals (block 86). In certain embodiments, the processor 38 may derive the exhaled tidal volume from one or more determined physiological parameters, such as the expiratory flow. For example, the processor 38 may integrate the expiratory flow over time to derive the exhaled tidal volume. It should be noted that the processor 38 may also be configured to confirm the occurrence of each breath. That is, the processor 38 may monitor the pressure and/or flow to determine the initiation and completion of each breath (e.g., a delivered inspiratory flow). For example, the processor 38 may be configured to calculate the first derivative of the flow, the first derivative of the pressure, the second derivative of the flow, and/or the second derivative of the pressure to determine the initiation and completion of each breath.

To determine whether a double-triggering event occurred between two breaths, the processor 38 may compare the exhaled tidal volume to a threshold. In one embodiment, the processor 38 may determine if the exhaled tidal volume is approximately equal to zero millimeters (block 88). It should be noted that any other suitable threshold volume for the exhaled tidal volume may be selected, and the processor 38 may determine that a double-triggering event occurred if the exhaled tidal volume is less than the threshold volume. Moreover, the processor 38 may be configured to monitor other parameters to detect double-triggering events, such as the expiratory time or the ratio of the exhaled tidal volume with respect to the inhaled tidal volume. For example, the processor 38 may determine that a double-triggering event occurred between two breaths if the expiratory time is less than an expiratory time threshold.

However, determining whether the exhaled tidal volume is approximately equal to zero millimeters may be advantageous as compared to setting a different minimum threshold volume. That is, a double-triggering event may result from a patient cough, a patient sigh, a patient yawn, inappropriate ventilator settings (e.g., inspiratory time, inhaled tidal volume, and/or respiratory rate), and/or a condition of the patient. A zero millimeter threshold may identify double-triggering events that may be caused by inappropriate ventilator settings, rather than coughs, sighs, and yawns. For example, the patient may transition to expiration without triggering a stacked breath, but the patient may cough, sigh, or yawn during the expiratory period, which may trigger a stacked breath. As such, this double-triggering event may not indicate that the patient may benefit from an adjustment in ventilator settings. In contrast, an exhaled tidal volume that is approximately equal to zero millimeters may indicate that the patient did not expire between the two breaths. Rather, it may indicate that the patient continued to inspire (e.g., to achieve a greater inhaled tidal volume and/or a longer inspiratory time), thus, triggering a stacked breath. Accordingly, determining whether the exhaled tidal volume is approximately equal to zero may be desirable to more accurately identify double-triggering events that may be more indicative of inappropriate ventilator settings.

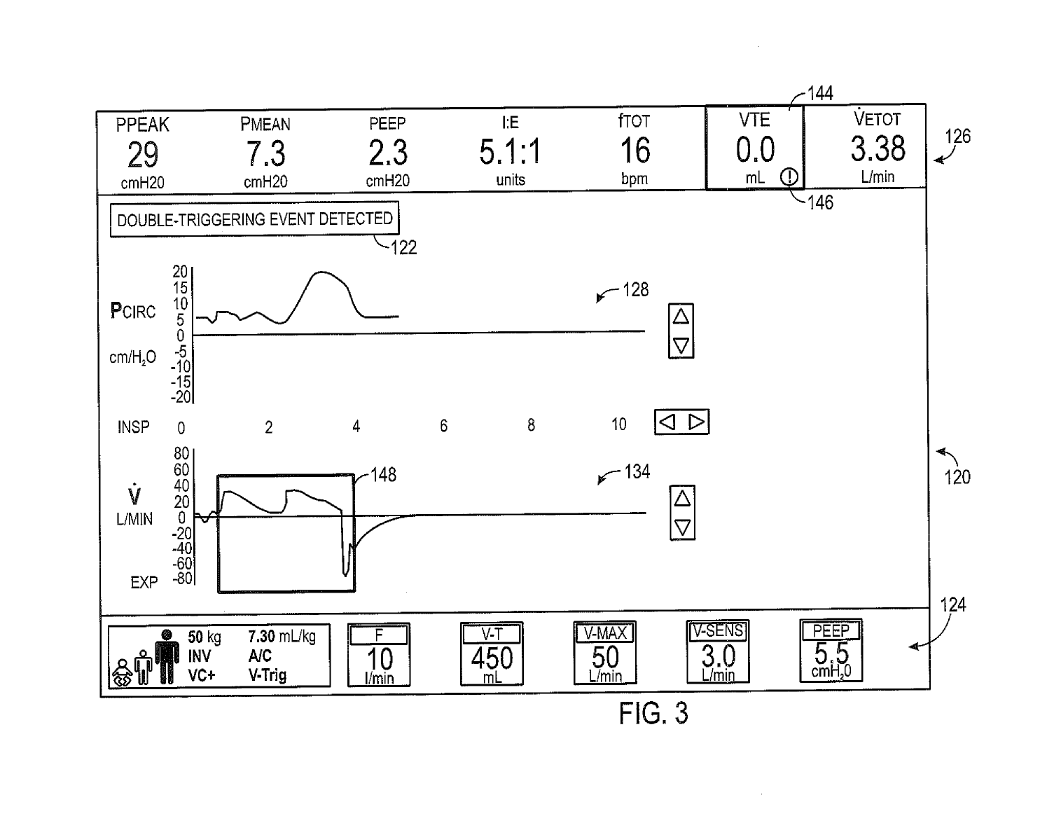

If the processor 38 determines that the exhaled tidal volume is approximately equal to zero millimeters, the processor 38 may cause the ventilator 12 to provide an indication of a double-triggering event detected (block 90). In certain embodiments, the processor 38 may additionally cause the accessory device 56 to provide the indication. The indication of a double-triggering event detected may be user-perceptible. For example, the processor 38 may cause the display 52 and/or a display of the accessory device 56 to display a visual indication, which may be textual, graphical, or any other suitable indication. Various embodiments of the indication of a double-triggering event detected will be described in more detail below with respect to FIGS. 3-6. Additionally or alternatively, the processor 38 may cause the speaker 54 and/or a speaker of the accessory device 56 to provide an audible indication, such as an alarm or a beep. In this manner, the ventilator 12 and/or the accessory device 56 may direct the caregiver's attention to the ventilator 12 and/or the accessory device 56, so that the caregiver may be aware that the patient experienced a double-triggering event and may benefit from a reassessment of his or her condition and treatment (e.g., ventilator settings and/or ventilator operating mode). Alternatively, if the exhaled tidal volume is not approximately equal to zero millimeters, the processor 38 may continue monitoring the signals from the sensors 48 (block 84).

Because a double-triggering event may occur as an isolated event, it may be desirable to also monitor the frequency of double-triggering events. That is, a double-triggering event may occur due to a cough, sigh, or yawn. Additionally, a double-triggering event may occur because the patient briefly desires a longer and/or larger breath. However, an isolated double-triggering event or a few intermittent double-triggering events may not necessarily indicate that the patient may benefit from an adjustment in ventilator settings and/or a different ventilator operating mode.

Accordingly, the method 80 may include determining whether a frequency of double-triggering is above a respective double-triggering frequency threshold (block 92). That is, more than one double-triggering frequency threshold may be inputted by the caregiver via the control inputs 46 and/or stored in the RAM 40, the ROM 42, and/or the mass storage device 44 to be utilized by the processor 38. In certain embodiments, it may be desirable to set a threshold for the number of double-triggering events within a predetermined period of time, a threshold for the percentage of double-triggering events out of the total number of breaths within a predetermined period of time, a threshold for the total number of double-triggering events, and/or a threshold for the total percentage of double-triggering events out of the total number of breaths. For example, the predetermined period of time may be between approximately 30 seconds and 300 seconds, 45 seconds and 240 seconds, 60 seconds and 180 seconds, 90 seconds and 120 seconds, or any other suitable time period. The processor 38 may determine whether the number of double-triggering events within the predetermined period of time is at least three, four, five, six, or any other suitable number of double-triggering events. Additionally or alternatively, the processor 38 may determine whether the percentage of double-triggering events out of the total number of breaths within the predetermined period of time is at least approximately 10 percent, 15 percent, 20 percent, 25 percent, 30 percent, or any other suitable percentage. It should be noted that any suitable frequency thresholds may be used.

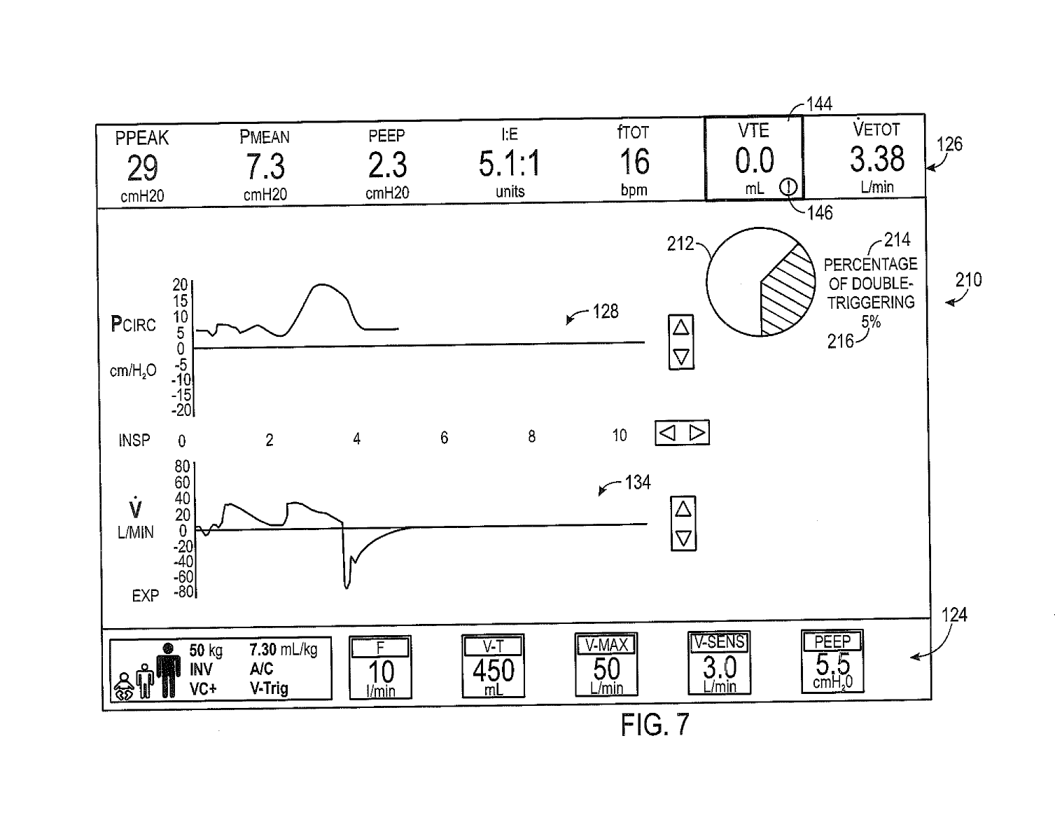

In response to determining that the frequency of double-triggering is above a respective frequency threshold, the processor 38 may cause the ventilator 12 to provide an indication of the frequency of double-triggering (block 94). In certain embodiments, the processor 38 may additionally cause the accessory device 56 to provide the indication. The indication of a double-triggering event detected may be user-perceptible. For example, the processor 38 may cause the display 52 and/or a display of the accessory device 56 to display a visual indication, which may be textual, graphical, or any other suitable indication. Various embodiments of the indication of the frequency of double-triggering will be described in more detail below with respect to FIGS. 7-10. Additionally or alternatively, the processor 38 may cause the speaker 54 and/or a speaker of the accessory device 56 to provide an audible indication, such as an alarm or a beep. In this manner, the ventilator 12 and/or the accessory device 56 may direct the caregiver's attention to the ventilator 12 and/or the accessory device 56, so that the caregiver may be aware of the severity of the patient's double-triggering and may consider actions to reduce the frequency of double-triggering.

As noted above, the display 52 may display various visual indications of a double-triggering event detected when the processor 38 determines that a double-triggering event has occurred, as well as visual indications of the frequency of double-triggering when the frequency exceeds a frequency threshold. Additionally, as noted above, the ventilator 12 may be configured to transmit data via the wireless transceiver 50. Thus, the various visual indications of minimal support detected may be displayed on the display 52 and/or a display of the accessory device 56 (e.g., a remote computer or a smart phone). Accordingly, while the embodiments described below in FIGS. 3-10 are described in the context of the display 52, it should be noted that the embodiments may be displayed on any suitable display, which may be external to the ventilator 12. Furthermore, it should be noted that the embodiments of the indication of a double-triggering event detected as described below with respect to FIGS. 3-6 may be utilized with embodiments of the indication of the frequency of double-triggering described below with respect to FIGS. 7-10 in any suitable combination.

For example, FIG. 3 is an illustration 120 of the display 52 including an indication of a double-triggering event detected 122. Additionally, the display 52 may display ventilator settings 124, calculated and/or derived physiological characteristics 126, a graph 128 of the pressure of the patient's respiratory circuit over time, and a graph 134 of the flow of the patient's respiratory circuit over time. As illustrated, the calculated and/or derived physiological characteristics 126 may include a value of the patient's exhaled tidal volume 144.

The indication of a double-triggering event detected 122 may be illustrated on the display 52 in any suitable means for conveying the indication to the caregiver. As illustrated, the indication of a double-triggering event detected 122 may be a textual indication. However, as will be described in more detail below, the indication of a double-triggering event detected 122 may also include a graphical representation of a double-triggering event. The indication of a double-triggering event detected 122 may be located below any of the calculated and/or derived physiological characteristics 126, near an alarm display, or any other suitable location. Additionally, the indication of a double-triggering event detected 122 may be displayed as a tab, a banner, a dialog box, or any other suitable type of display. In certain embodiments, the indication of a double-triggering event detected 122 may be displayed in the same font, color, shading, and/or size as the value of the patient's exhaled tidal volume 144. Furthermore, the processor 38 may be configured to alter the font, color, shading, and/or size of the indication of a double-triggering event detected 122 and the value of the patient's exhaled tidal volume 144 from the other elements on the display 52. Additionally, the indication of a double-triggering event detected 122 and/or the value of the patient's exhaled tidal volume 144 may also include a symbol 146, such as an exclamation point, an asterisk, a star, a stop sign, or any other suitable symbol.

Furthermore, to assist the caregiver in graphically identifying the detected double-triggering event, the processor 38 may be configured to provide a graphical indicator 148 to highlight the double-triggering event on the graph 134 of the flow of the patient's respiratory circuit. The double-triggering even may be more easily recognized on the graph 134 of the flow of the patient's respiratory circuit, and thus, in certain embodiments, the graphical indicator 148 may be displayed on only the graph 134 to reduce clutter on the display 52. However, the graphical indicator 148 may additionally or alternatively be displayed on the graph 128 of the pressure of the patient's respiratory circuit. As illustrated, the graphical indicator 148 may be a box surrounding the double-triggering event. However, any other suitable shape may be used. Furthermore, the graphical indicator 148 may alter the color, style, and/or thickness of the line of the graph 134 corresponding to the double-triggering event. In certain embodiments, the graphical indicator 148 may flash.

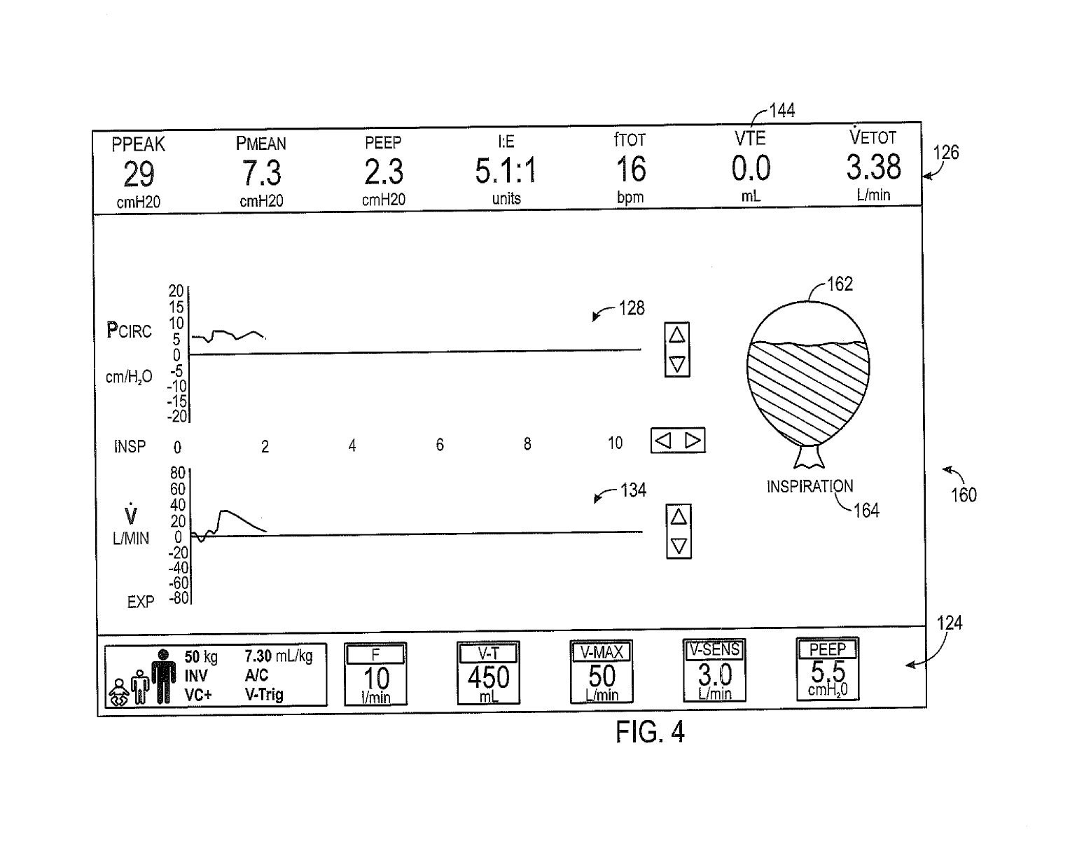

Because the double-triggering event may be difficult to graphically detect on the graph 128 and/or the graph 134, it may be desirable to provide a graphical representation of the double-triggering event. Furthermore, it may be desirable to provide a graphical representation indicating the inspiration of the patient for each breath, such that when the patient experiences a double-triggering event, the graphical representation may change to indicate the double-triggering event. For example, FIG. 4 is an illustration 160 of the display 52 including a graphical representation 162 of the patient's inspiration. As illustrated, the graphical representation 162 may also include a label 164, such as a textual message (e.g., “inspiration”). The graphical representation 162 may be a balloon, a person's lungs, a triangle, a rectangle, a circle, or any other suitable shape. The graphical representation 162 may be configured to fill (e.g., with shading and/or a color) with respect to the elapsed inspiratory time or the inhaled tidal volume for each breath. That is, the graphical representation 162 may fill with respect to the elapsed inspiratory time in embodiments in which the inspiratory and expiratory modules 26 and 28 are configured to transition to expiration based on a preset inspiratory time. Similarly, the graphical representation 162 may fill with respect to the inhaled tidal volume in embodiments in which the inspiratory and expiratory modules 26 and 28 are configured to transition to expiration based on a preset inhaled tidal volume.

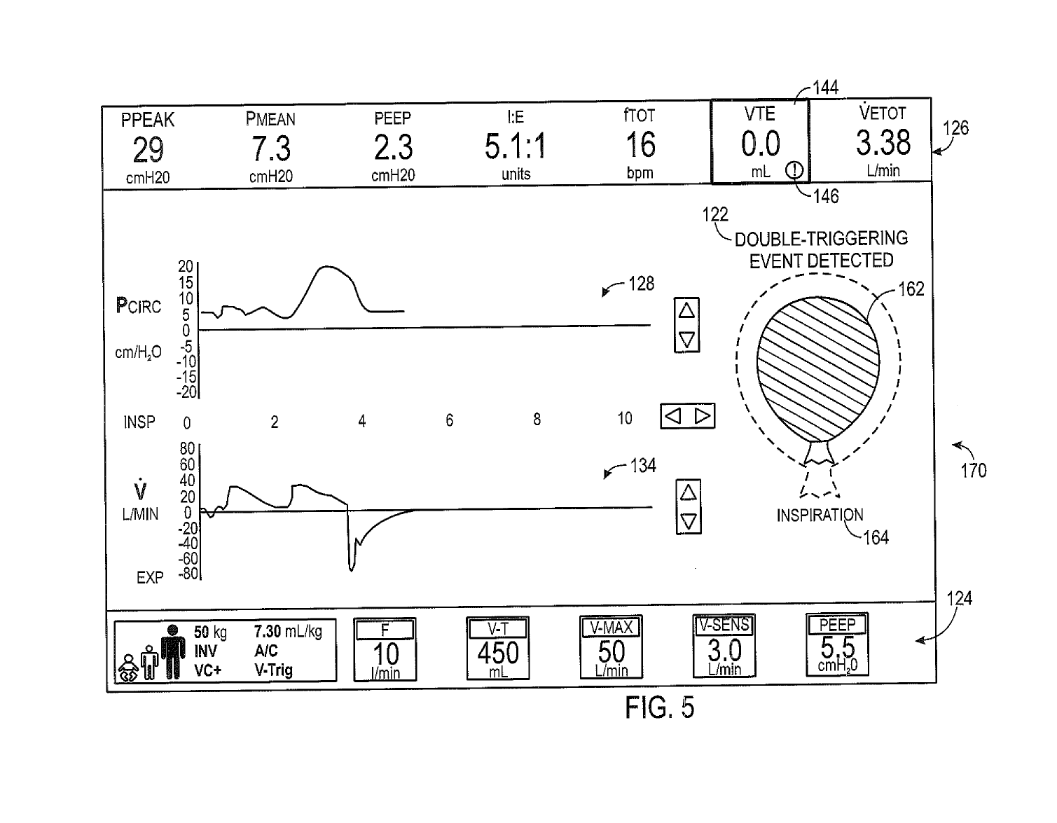

Accordingly, if the preset inspiratory time or preset tidal volume is reached, the graphical representation 162 may be illustrated as entirely filled. If the processor 38 detects a double-triggering event, the processor 38 may be configured to alter the appearance of the filled graphical representation 162. For example, FIG. 5 is an illustration 170 of the display 52 including the indication of a double-triggering event detected 122 and the graphical representation 162 of the patient's inspiration during a double-triggering event. In particular, as illustrated by the dashed lines, the graphical representation 162 of a double-triggering event may increase in size during the double-triggering event (i.e., an enlarged version of the graphical representation 162 illustrated in FIG. 4). In this manner, the enlarged graphical representation 162 may represent an expansion of the patient's lungs caused by the double-triggering event. Additionally, the processor 38 may be configured to alter the color, shading, texture, and/or line quality of the graphical representation 162 and/or cause the graphical representation 162 to flash during the double-triggering event.

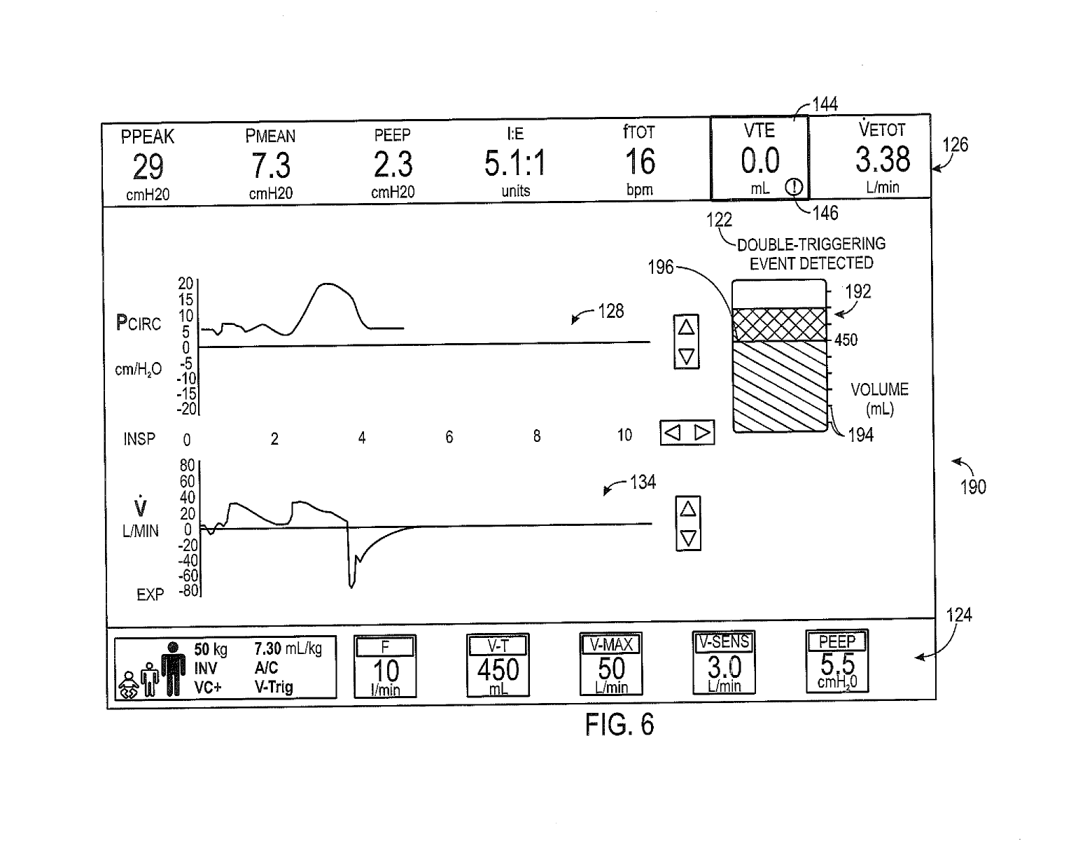

While the graphical representation 162 may provide an indication of the double-triggering event, it may not provide information regarding the degree of the double-triggering event. Accordingly, FIG. 6 is an illustration 190 of the display 52 including the indication of a double-triggering event detected 122 and a graphical representation 192 of the patient's inhaled tidal volume. The graphical representation 192 may be displayed as a cylinder, a triangle, a rectangle, a circle, a balloon, a person's lungs, or any other suitable shape. As illustrated, the graphical representation 192 may include one or more tick marks 194 corresponding to an inhaled tidal volume. The graphical representation 192 may also include a threshold line 196 corresponding to a target inhaled tidal volume (i.e., the preset inhaled tidal volume). For example, the caregiver may set the target inhaled tidal volume at 450 milliliters or any other suitable target inhaled tidal volume using the control inputs 46. Thus, the graphical representation 192 may be user-configurable. The graphical representation 192 may be configured to fill (e.g., with shading and/or a color) with respect to the inhaled tidal volume, as described above with respect to FIGS. 4 and 5. However, if the processor 38 detects a double-triggering event, the double-triggering event may be represented on the graphical representation 192 as the processor 38 may be configured to fill the graphical representation 192 above the threshold line 196 with a different shading and/or color. In this manner, the caregiver may more easily assess the increase in the inhaled tidal volume resulting from the double-triggering event.

As described above, it may be desirable to additionally provide an indication of the frequency of double-triggering to alert the caregiver to the severity of the double-triggering. Accordingly, FIG. 7 is an illustration 210 of the display 52 including a graphical representation 212 of the frequency of double-triggering. The graphical representation 212 may be illustrated as a circle, a triangle, a rectangle, a balloon, a patient's lungs, or any other suitable shape. The graphical representation 212 may be configured to change (e.g., fill and/or empty with shading and/or a color) with respect to the percentage of double-triggering events within a predetermined period of time or the number of double-trigging events within a predetermined period of time. Specifically, the processor 38 may be configured to set a predetermined period of time, which may be inputted by the caregiver via the control inputs 46. The processor 38 may calculate the percentage of double-triggering events out of the total number of breaths within the predetermined period of time or the number of double-triggering events within the predetermined period of time. Accordingly, the processor 38 may be configured to cause the graphical representation 212 to change (e.g., fill and/or empty) with respect to the calculated percentage. In one embodiment, the graphical representation 212 may represent the total percentage of double-triggering (i.e., not within a predetermined period of time). Furthermore, the graphical representation 212 may represent a frequency threshold. For example, the caregiver may set the frequency threshold via the control inputs 46 to approximately fifteen percent. Thus, if the percentage of double-triggering is approximately five percent, the processor 38 may be configured to fill approximately one-third of the graphical representation 212. Accordingly, the graphical representation 212 may be configured with any suitable frequency threshold, which may be inputted by the caregiver. Additionally, for to provide a more readable indication, the illustration 210 may also include a label 214 (e.g., “percentage of double-triggering”) and a numerical indication 216 of the calculated percentage of double-triggering (e.g., “5%”).

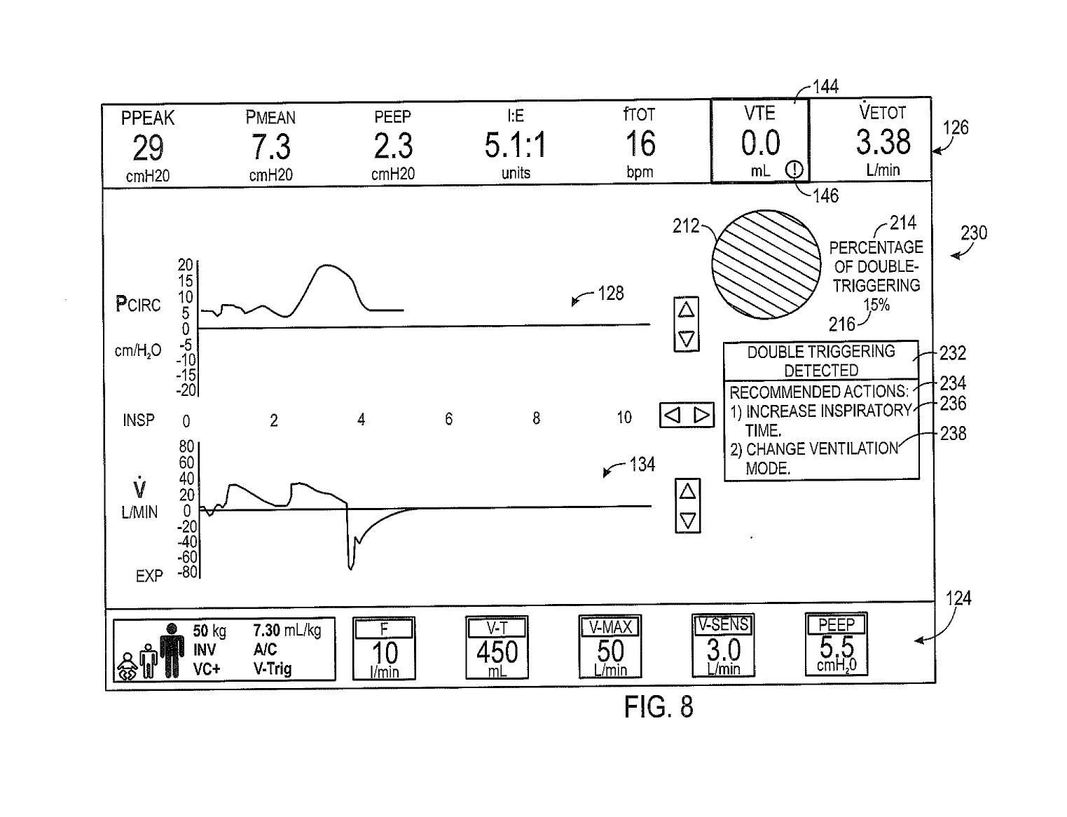

Accordingly, if the percentage of double-triggering reaches or exceeds the preset frequency threshold, the graphical representation 212 may be illustrated as entirely filled. For example, FIG. 8 is an illustration 230 of the display 52 including the filled graphical representation 212. Accordingly, the numerical indication 216 may indicate the frequency threshold (e.g., “15%”) or may indicate a percentage of double-triggering above the frequency threshold (e.g., “35%”), if the calculated percentage of double-triggering exceeds the frequency threshold. In certain embodiments, the graphical representation 212 may be configured to increase in size, change color and/or shading, and/or flash if the percentage of double-triggering exceeds the frequency threshold.

Additionally, the illustration 230 may include an indication of double-triggering detected 232. It should be noted that the indication of double-triggering detected 232 may be provided when a frequency of double-triggering exceeds a respective frequency threshold, while the indication of a double-triggering event detected 212 may be provided for each detected double-triggering event. As illustrated, the indication of double-triggering detected 232 may be a textual indication. Additionally, the indication of double-triggering detected 232 may be displayed as a tab, a banner, a dialog box, or any other suitable type of display. The indication of double-triggering detected 232 may be located below the graphical representation 212, below any of the calculated and/or derived physiological characteristics 126, near an alarm display, or in any other suitable location. In certain embodiments, the indication of double-triggering detected 232 may be displayed in the same font, color, shading, and/or size as the value of the patient's exhaled tidal volume 144 and/or the indication of a double-triggering event detected 212. Furthermore, the processor 38 may be configured to alter the font, color, shading, and/or size of the indication of double-triggering detected 232 and the value of the patient's exhaled tidal volume 144 from the other elements on the display 52.

To assist the caregiver in reducing the frequency of double-triggering, the indication of double-triggering detected 232 may also include a list of recommended actions 234. In particular, the list of recommended actions 234 may include one or more actions that, when performed, may reduce the frequency of double-triggering. Additionally, the actions in the list of recommended actions 234 may be listed in order of the likelihood that action may reduce the frequency of double-triggering. For example, the list of recommended actions 234 may include a recommendation to increase the preset inspiratory time 236. As will be appreciated, the list of recommended actions 234 may include a recommendation to adjust any suitable ventilation settings 122, such as the inhaled tidal volume or the respiratory rate. The list of recommended actions 234 may also include a recommendation to change the ventilation operating mode 238. That is, if adjusting one or more ventilation settings 122 does not sufficiently decrease the frequency of double-triggering, the caregiver may consider switching the ventilation operating mode (e.g., from a volume support mode to a pressure support mode).

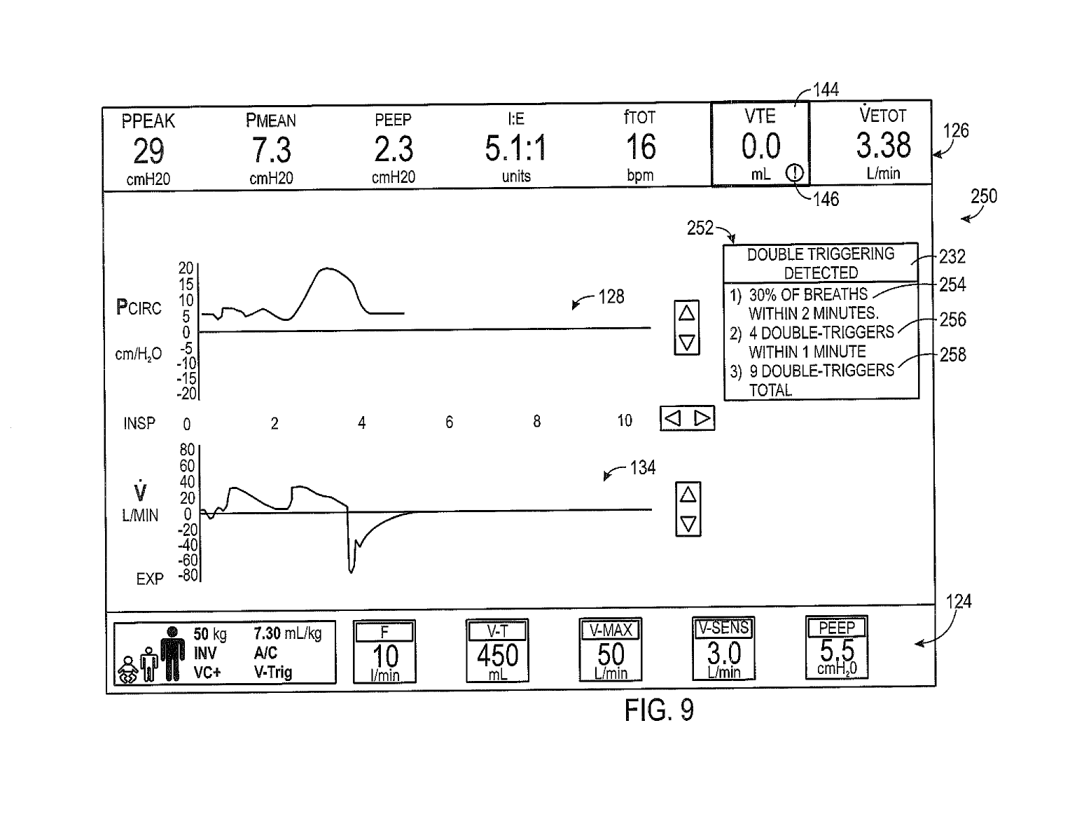

As described above, the processor 38 may be configured to determine one or more values of the frequency of double-triggering. For example, the processor 38 may calculate the percentage of double-triggering events out of the total number of breaths within a predetermined period of time, the number of double-triggering events out of the total number of breaths within a predetermined period of time, and/or the total number of double-triggering events. Accordingly, in certain embodiments, it may be desirable to provide the caregiver with the one or more values of the frequency of double-triggering. For example, FIG. 9 is an illustration 250 of the display 52 including the indication of double-triggering detected 232 and a list of values of the frequency of double-triggering 252. In certain embodiments, the list may include the percentage of double-triggering events out of the total number of breaths within a predetermined period of time 254, the number of double-triggering events out of the total number of breaths within a predetermined period of time 256, and the total number of double-triggering events 258. As illustrated, certain values of the frequency of double-triggering may use different predetermined periods of time and/or may use more than one predetermined period of time. For example, the percentage of double-triggering events out of the total number of breaths within a predetermined period of time 254 may use a period of time of approximately thirty seconds and approximately two minutes. The number of double-triggering events out of the total number of breaths within a predetermined period of time 256 may use a period of time of approximately one minute and approximate three minutes. However, any other suitable period of time may be used.

As described in detail above, providing the caregiver with the indication of a double-triggering event detected 122 and/or the indication of double-triggering detected 232 may enable the caregiver to recognize double-triggering events and to assess the frequency of double-triggering more readily. Furthermore, the indication of a double-triggering event detected 122 and/or the indication of double-triggering detected 232 may alert the caregiver that the patient may benefit from a reassessment of the patient's condition and/or treatment. To assist the caregiver in reassessing the patient, the ventilator 12 may also be configured to provide historical data relating to each detected double-triggering event.

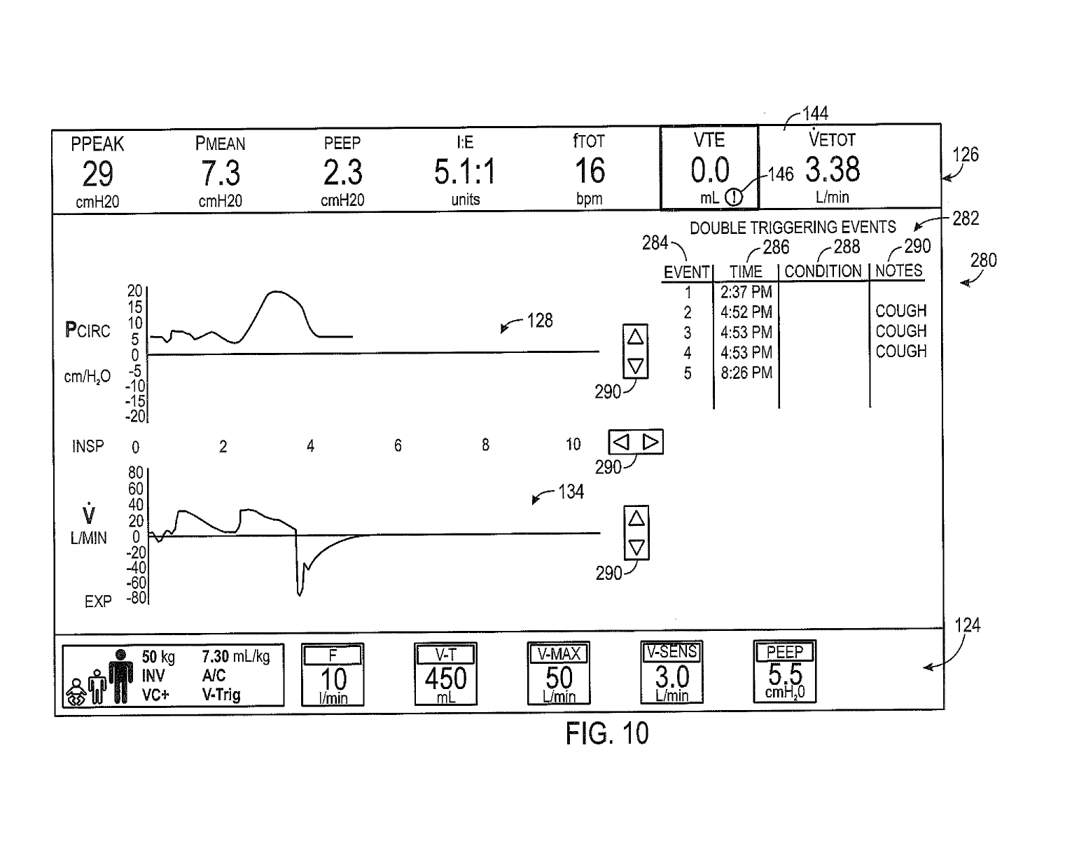

Accordingly, FIG. 10 is an illustration 280 of the display 52 including a table 282 of double-triggering events. The table 282 may be located near the indication of a double-triggering event detected 122, near the indication of double-triggering detected 232, below any of the calculated and/or derived physiological characteristics 126, near an alarm display, or in any other suitable location. Additionally, the table 282 may be configured to appear when the processor 38 first detects a double-triggering event. Alternatively, the table 282 may be configured to appear when the indication of a double-triggering event detected 122, the indication of double-triggering detected 232, the value of the exhaled tidal volume 144, and/or the symbol 146 is selected by the caregiver (e.g., via the control inputs 46 or a touch-screen display 52).

The processor 38 may be configured to collect data relating to each double-triggering event, to store the data in the RAM 40, the ROM 42, and/or the mass storage device 44, and to form and/or update the table 282 using the stored data. The table 282 may include an event column 284, a time column 286, a condition column 288, and a notes column 290. However, any suitable column may be incorporated into the table 282. The event column 284 may numerically label each detected double-triggering event and may list the double-triggering events in the order in which they occurred. The time column 286 may list the time that each double-triggering event occurred. The time column 286 may additionally include the date, if desired. Alternatively, the table 282 may incorporate a date column. The condition column 288 may provide data relating to the patient's condition during or surrounding (i.e., within a preset period of time) the double-triggering event. The data relating to the patient's condition may be collected by the ventilation system 10 and/or one or more external medical devices (e.g., a pulse oximeter, an electrocardiography device, an end-tidal carbon dioxide (EtCo₂) monitor, and/or an electroencephalogy (EEG) device). In certain embodiments, the condition column 288 may provide data relating to the patient's condition if the data is outside of normal range and/or if the data is determined to be related to the double-triggering event.

The notes column 284 may enable the caregiver to view any notes that were inputted into the ventilator 12 regarding the detected double-triggering event. That is, if the caregiver is present during the double-triggering event, the caregiver may input any relevant information via the control inputs 46 that may assist the caregiver in the reassessment of the patient's condition and/or treatment. For example, the caregiver may make a note that a double-triggering event may have occurred because the patient was coughing, sighing, or yawning. Indeed, as described above, double-triggering events may occur due to a patient cough, sigh, or yawn, but may not necessarily indicate that the patient may benefit from an adjustment in the ventilator settings 122. As such, if the caregiver is present, it may be desirable to note which double-triggering events may have occurred because the patient was coughing, sighing, or yawning.

Additionally, it may be desirable to enable the caregiver to view historical data on the graph 128 of the pressure of the patient's respiratory circuit and/or on the graph 134 of the flow of the patient's respiratory circuit. In particular, the caregiver may wish to view pressure and/or flow of the patient's respiratory circuit during the time surrounding a double-triggering event. Accordingly, in certain embodiments, the processor 38 may cause the graph 128 and/or the graph 134 to display the pressure waveform and/or the flow waveform at the time corresponding to a double-triggering event selected on the table 282. For example, the caregiver may select the first double-triggering event on the table 282 (i.e., event 1) using the control inputs 46 or by touching its corresponding row (i.e., the row for event 1) on the table 282. In response to the caregiver's selection, the processor 38 may cause the graph 128 and/or the graph 134 to display the pressure waveform and/or the flow waveform at 2:37 PM (i.e., the time that event 1 occurred). It should be noted that the graphs 128 and 134 may be configured to center their respective waveforms at the designated time such that the caregiver may view a portion of the waveforms surrounding the designated double-triggering event. Additionally or alternatively, the caregiver may scroll through the graphs 128 and 134 using the control arrows 290 to view historical data of the graphs 128 and 134.

The above embodiments describe a ventilation system configured to detect double-triggering events. In certain embodiments, the ventilation system may detect double-triggering events by determining whether an exhaled tidal volume between two inspiratory breaths is approximately equal to zero millimeters. In further embodiments, the ventilation system may be configured to provide an indication of a double-triggering event detected, which may be a textual indication and/or a graphical representation.

Further, the above embodiments describe the use of the ventilation system to determine a frequency of double-triggering. In certain embodiments, the ventilation system may be configured to determine the number of double-triggering events within a predetermined period of time and/or the percentage of double-triggering events out of a total number of breaths within a predetermined period of time. In further embodiments, the ventilation system may be configured to provide an indication of double-triggering detected if a determined frequency of double-triggering is above a respective frequency threshold. In some embodiments, the ventilation system may be configured to provide the indication of double-triggering detected as a textual indication, a graphical representation, and/or a list of the determined values of the frequency of double-triggering.

The disclosed embodiments may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A ventilator, comprising:
a processor configured to calculate a frequency of double-triggering events of a patient and to determine whether the frequency of the double-triggering events exceeds a frequency threshold; and
a display configured to provide a graphical representation of the frequency of double-triggering.

2. The ventilator of claim 1, wherein the display is configured to provide an indication of detected double-triggering when the processor determines that the frequency of the double-triggering events exceeds the frequency threshold.

3. The ventilator of claim 2, wherein the indication of detected double-triggering comprises a textual indication or a number associated with the frequency of the double-triggering events.

4. The ventilator of claim 1, wherein the graphical representation is configured to change based at least in part upon the frequency of the double-triggering events.

5. The ventilation system of claim 4, wherein the graphical representation is configured to fill when the frequency of the double-triggering events increases.

6. The ventilator of claim 5, wherein the display is configured to provide the graphical representation as entirely filled when the processor determines that the frequency of the double-triggering events exceeds the frequency threshold.

7. The ventilator of claim 1, wherein the frequency of the double-triggering events comprises a percentage of double-triggering events out of a total number of breaths within a predetermined period of time.

8. The ventilator of claim 1, wherein the processor is configured to detect a double-triggering event, and wherein the display is configured to provide an indication of a detected double-triggering event.

9. The ventilator of claim 8, wherein the processor is configured to detect the double-triggering event based at least in part upon a determination that an exhaled tidal volume of the patient is approximately equal to zero millimeters.

10. The ventilator of claim 7, wherein the indication of the detected double-triggering event comprises a textual or a graphical indication.

11. A method, comprising:
calculating a frequency of double-triggering events of a patient via a processor of a ventilator; and
providing a graphical representation of the frequency of the double-triggering events on a display of the ventilator.

12. The method of claim 11, comprising:
determining, via the processor, whether the frequency of the double-triggering events exceeds a frequency threshold; and
providing an indication of detected double-triggering when the frequency of the double-triggering events exceeds the frequency threshold on the display.

13. The method of claim 11, comprising detecting, via the processor, one or more double-triggering events, wherein detecting the one or more double-triggering events is based at least in part upon a determination that an exhaled tidal volume of the patient is approximately equal to zero milliliters.

14. The method of claim 11, wherein calculating the frequency of the double-triggering events comprises calculating a percentage of double-triggering events out of a total number of breaths within a predetermined period of time.

15. The method of claim 11, comprising changing the graphical representation based at least in part upon the frequency of the double-triggering events.

16. The method of claim 15, comprising filling the graphical representation when the frequency of the double-triggering events increases.

17. A ventilation system, comprising:
one or more sensors configured to generate one or more signals representative of a respiratory function of a patient;
a processor configured to receive the one or more signals and to calculate a frequency of double-triggering events of the patient based at least in part upon the one or more signals; and
a display configured to provide a graphical representation of the frequency of double-triggering.

18. The ventilation system of claim 17, wherein the processor is configured to determine whether the frequency of the double-triggering events exceeds a frequency threshold, and wherein the display is configured to provide an indication of detected double-triggering when the processor determines that the frequency of the double-triggering events exceeds the frequency threshold.

19. The ventilation system of claim 17, comprising an accessory device configured to wirelessly communicate with the processor, wherein the accessory device comprises the display.

20. The ventilation system of claim 17, wherein the processor is configured to detect a double-triggering event based at least in part upon a determination that an exhaled tidal volume of the patient is approximately equal to zero millimeters.
